(19) United States
(12) Patent Application Publication
(10) Pub. No.: US 2021/0196619 A1
(43) Pub. Date: Jul. 1, 2021

(54) COMPOSITION COMPRISING ABIES SIBIRICA OIL FOR STRENGTHENING SKIN BARRIER

(71) Applicant: Amorepacific Corporation, Seoul (KR)

(72) Inventors: Euidong Son, Yongin-si (KR); Hyungsu Kim, Yongin-si (KR); Jihyun Kim, Yongin-si (KR); Hyoung June Kim, Yongin-si (KR); Byeongbae Jeon, Yongin-si (KR); Ji Young Choi, Yongin-si (KR); Tae Ryong Lee, Yongin-si (KR)

(73) Assignee: Amorepacific Corporation, Seoul (KR)

(21) Appl. No.: 17/197,526

(22) Filed: Mar. 10, 2021

Related U.S. Application Data
(63) Continuation-in-part of application No. PCT/KR2019/013577, filed on Oct. 16, 2019.

(30) Foreign Application Priority Data
Oct. 16, 2018 (KR) 10-2018-0123302

(51) Int. Cl. A61K 8/9767; A61K 8/04; A61K 9/00; A61K 9/06; A61K 36/15; A61Q 19/08; A61Q 19/00; A23L 33/105

(52) U.S. Cl. CPC A61K 8/9767; A61K 8/04; A61K 9/0014; A23L 33/105; A61K 36/15; A61Q 19/08; A61Q 19/007; A61K 9/06

(57) ABSTRACT

One aspect of the present disclosure relates to a composition for enhancing the skin barrier containing Abies sibirica oil as an active ingredient, wherein Abies sibirica oil reacts with the fragrance receptor OR6M1 present in epidermal cells to enhance the skin barrier, restore the damaged skin barriers and enhance the skin moisturizing.

### CROSS-REFERENCE TO RELATED APPLICATIONS

This application is a Continuation-in-Part of International Application No. PCT/KR2019/013577 filed Oct. 16, 2019, which claims priority under U.S.C. § 119(a) to Koran Patent Application No. 10-2018-0123302 filed on Oct. 16, 2018.

### TECHNICAL FIELD

One aspect of the present disclosure relates to a skin barrier enhancing composition comprising Abies sibirica oil and its use in a method of enhancing skin barrier function.

### BACKGROUND ART

The skin plays a very important role as a barrier function to protect a subject from the outside. The barrier function is a protective function that protects against various external stimuli such as chemicals, air pollutants, dry environments, UV rays, etc. and prevents excessive emission of body moisture through the skin, and such a protective function may be maintained only when the stratum corneum (horney layer) consisting of keratinocytes is normally formed. Such skin histologically consists of three layers of epidermis, dermis, and subcutis, and among them, the epidermis exists at the outermost of the skin, plays the most important role in terms of skin aging, and accordingly, becomes a subject of the most intensive research even in terms of skin beauty.

Even in the epidermis, components consisting of the stratum corneum as the outermost layer are corneocytes and skin lipids, wherein the skin lipids are responsible for a barrier function of the skin, and such a barrier function of the skin may be an important function to protect the skin from external harmful substances by regulating cell division and differentiation of the stratum corneum, prevent leakage of substances in the body, and prevent evaporation of skin moisture.

Among the skin lipids, a main lipid serving as the skin barrier function is the lipid produced by the differentiation of keratinocytes in the epidermis, and these lipids fill spaces between differentiated corneocytes, and thus play a role in imparting intercellular binding. The lipid mainly consists of ceramide, cholesterols and free fatty acids, which account for about 40% to 65%, and 10% and 25% of the total lipid in the lipid layer, respectively. In addition, the lipid has a composition in which small amounts of phytosphingosine and sphingosine are present together.

Meanwhile, it is known that fragrance receptors present in the nose are also present even in the skin as well as various organs. At this time, unlike the nose, it has been known that functions of fragrance receptors in organs and skin are different from those in the nose. Among the fragrance receptors, olfactory receptor family 6 subfamily M member 1 (OR6M1) can be used as a biomarker for diagnosing or evaluating a skin barrier function when expressed in epidermal cells. However, the fragrance that reacts with the fragrance receptor has not been known until now, and no reports have been made on a skin barrier enhancing function or a moisturizing effect using the reaction with the fragrance receptor.

### DISCLOSURE

### Technical Problem

Therefore, the present inventors found that there was an effect of enhancing a skin barrier or restoring a damaged skin barrier by reacting Abies sibirica oil with a fragrance receptor OR6M1 capable of diagnosing or evaluating a skin barrier function, and then completed one aspect of the present disclosure.

Accordingly, an object of one aspect of the present disclosure is to provide a skin barrier enhancing composition comprising Abies sibirica oil as an active ingredient.

### Technical Solution

In order to achieve the above object, one aspect of the present disclosure provides a skin barrier enhancing composition comprising Abies sibirica oil as an active ingredient.

### Advantageous Effects

The composition of one aspect of the present disclosure has a skin barrier enhancing effect.

### DESCRIPTION OF DRAWINGS

FIG. 1 is a photograph of observing the cell membrane expression of OR6M1 in HEK 293 cells.

FIG. 2 is a graph of measuring a cAMP concentration according to DMSO treatment.

FIG. 3 is a graph of measuring a cAMP concentration according to a concentration of Abies sibirica oil.

FIG. 4 is a graph of measuring the degree of restoring a skin barrier damage caused by ultraviolet rays in Experimental Example 3.

### BEST MODE

Hereinafter, one aspect of the present disclosure will be described in more detail.

One aspect of the present disclosure relates to a skin barrier enhancing composition comprising Abies sibirica oil as an active ingredient.

The Abies sibirica oil is extracted from Abies sibirica, and may be oil extracted from the leaves, roots, stems, fruits, or mixtures thereof of the Abies sibirica, but is not limited thereto.

The extraction of the Abies sibirica oil is not particularly limited as long as it is a method known in the art, and as an embodiment, the Abies sibirica oil may be obtained by using steam distillation. The steam distillation refers to a method of collecting useful ingredients by distilling through steam.

Specifically, when the Abies sibirica is placed in a container designed to allow water vapor to pass through the container, the cell walls of the Abies sibirica are broken down, and the essence and water vapor between the cell walls are collected. After the essence and water vapor are cooled while pass through pipes, the water vapor is separated into a distillate (aqueous solution ingredient) and the essence into oil (fat-soluble ingredient). At this time, since the oil is light and exists in an upper layer, the Abies sibirica oil may be obtained through separation of the upper layer.

In addition, the Abies sibirica oil has about 34 kinds of main ingredients, and among them, has a main composition consisting of 20 wt % to 30 wt % of campene, 10 wt % to 20 wt % of delta-3-carene, 10 wt % to 20 wt % of alpha-pinene, and 1 wt % to 5 wt % of myrcene.

The Abies sibirica oil has a pine fragrance and is characterized by reacting with a human OR6M1 gene, which is a fragrance receptor present in the skin.

The olfactory receptor family 6 subfamily M member 1 (OR6M1) gene is a fragrance receptor, encodes an olfactory receptor 6M1 protein of olfactory cells, and an NCBI accession ID of the OR6M1 gene is NM_001005325.1.

In addition, the OR6M1 gene, which is the fragrance receptor, can be used as a biomarker for diagnosing or evaluating a skin barrier function when expressed in epidermal cells.

The Abies sibirica oil of one aspect of the present disclosure reacts with the fragrance receptor OR6M1 present in epidermal cells, and may enhance the skin barrier function through the reaction.

More specifically, the fragrance of the Abies sibirica oil reacts with the fragrance receptor OR6M1 present in epidermal cells, and may enhance the skin barrier through the reaction.

In one aspect of the present disclosure, the skin barrier enhancing effect also means including an effect of restoring a damaged skin barrier.

Therefore, the composition of one aspect of the present disclosure may exhibit the effect of enhancing the skin barrier function and restoring the damaged skin barrier.

The skin barrier (stratum corneum) consists of dead corneocytes and intercellular lipids, and plays a key role in skin health as a skin protection layer which protects the skin from external stimuli and prevents moisture from evaporating from the skin.

Therefore, the composition of one aspect of the present disclosure may also exhibit a skin moisturizing enhancing effect.

An embodiment of the present disclosure is directed to a use of the composition in a method of improving skin condition or aesthetic appearance of skin of a subject. The subject may include a human. The method for improving the condition or aesthetic appearance of subject skin comprises topically applying to skin in need thereof an effective amount of a topical composition, wherein said improvement is selected from the group consisting of: (a) treatment, reduction, and/or prevention of fine lines or wrinkles, (b) improvement in skin thickness, plumpness, and/or firmness; (c) improvement in skin suppleness and/or softness; (d) improvement in skin tone, radiance, and/or clarity; (e) improvement in skin barrier repair and/or function; (f) improvement in appearance of skin contours; (g) improvement in skin moisturization; or any combination thereof.

The Abies sibirica oil of one aspect of the present disclosure may be included in an amount of 0.01 wt % to 10 wt %. Specifically, the content of the Abies sibirica oil is 0.01% by weight or more, and 1% by weight or less, 10% by weight or less based on the total weight of the composition.

When the Abies sibirica oil is included in an amount of 0.01 wt % to 1 wt %, the amount is not only appropriate to exhibit the intended effect of one aspect of the present disclosure, but also may satisfy both the stability and safety of the composition, and is preferable even in terms of cost effectiveness.

The skin barrier function enhancing composition of one aspect of the present disclosure may be a topical composition such as a cosmetic or dermatological composition.

The topical composition may be provided in all formulations suitable for topical application. For example, the cosmetic composition may be provided in formulations, such as a liquid, a cream, a lotion, an emulsion obtained by dispersing an oil phase in an aqueous phase, an emulsion obtained by dispersing an aqueous phase in an oil phase, a suspension, a solid, a gel, a powder, a paste, a patch, a mask, a towelette, a wax-based stick, a foam, or an aerosol composition. The composition in such formulations may comprise a cosmetically or dermatologically acceptable carrier and may be prepared according to general conventional methods in the art. A cosmetically or dermatologically acceptable carrier that can be used in the present topical compositions include, but are not limited to, one or more aqueous systems, glycerins, C1-4 alcohols, fatty alcohols, fatty ethers, fatty esters, polyols, glycols, vegetable oils, mineral oils, liposomes, laminar lipid materials, silicone oils, water or any combinations thereof.

In the present disclosure, the carrier may be in the form of an aqueous phase, an oil phase, a gel, a wax-in-water emulsion, a silicone-in-water emulsion, a water-in-silicone, an oil-in-water emulsion, or a water-in-oil emulsion. The aqueous phase is a mixture of one or more water soluble or water dispersible ingredient, which can be liquid, semi-solid or solid at room temperature (approximately 25° C.).

In addition to the above-described substances, the cosmetic composition may contain other ingredients capable of giving a synergistic effect on the skin barrier enhancing effect within a range that does not impair the skin barrier enhancing effect. The cosmetic composition according to one aspect of the present disclosure may contain a substance selected from the group consisting of vitamins, polymer peptides, molecular polysaccharides, and sphingo lipids. In addition, the cosmetic composition according to one aspect of the present disclosure may include a moisturizing agent, an emollient agent, a surfactant, an ultraviolet absorber, a preservative, a bactericide, an antioxidant, a pH adjuster, an organic pigment, an inorganic pigment, a fragrance, a cooling agent or a limiting agent. The mixing amount of the ingredients can be easily selected by those skilled in the art within a range that does not impair the object and effect of one aspect of the present disclosure.

Further, the skin barrier function enhancing composition of one aspect of the present disclosure may be a pharmaceutical composition.

The pharmaceutical composition may be formulated as an oral or parenteral preparation in a solid, semi-solid or liquid form by adding a commercially available inorganic or organic carrier using the composition as an active ingredient.

The preparations for oral administration include tablets, pills, granules, soft and hard capsules, fine granules, powders, emulsions, syrups, pellets, and the like. In addition, the preparations for parenteral administration may include injections, drops, ointments, lotions, sprays, suspensions, emulsions, and suppositories. In order to formulate the active ingredient of one aspect of the present disclosure, the active ingredient may be easily formulated according to conventional methods, and surfactants, excipients, coloring agents, spices, preservatives, stabilizers, buffers, suspensions, and other commercially available auxiliary agents may be suitably used.

The pharmaceutical composition according to one aspect of the present disclosure has an excellent effect of enhancing the skin barrier and may be usefully used in the treatment and prevention of skin diseases caused by damage to the skin barrier. Skin diseases caused by the damage to the skin barrier include atopic dermatitis, xeroderma, psoriasis, ichthyosis, acne, and the like, but are not limited thereto.

The pharmaceutical composition may be administered orally, parenterally, rectally, topically, transdermally, intravenously, intramuscularly, intraperitoneally, subcutaneously, or the like.

In addition, the dosage of the active ingredient will vary depending on the age, sex, and weight of a subject to be treated, a specific disease or pathological condition to be treated, the severity of the disease or pathological condition, the route of administration, and the judgment of a prescriber. The determination of the dosage based on these factors is within the level of those skilled in the art. A general dosage is 0.001 mg/kg/day to 2000 mg/kg/day, specifically 0.5 mg/kg/day to 1500 mg/kg/day.

Further, the skin barrier function enhancing composition of one aspect of the present disclosure may be a food composition.

In addition to the above-described ingredients, the food composition according to one aspect of the present disclosure may further include other ingredients having an effect of enhancing the skin barrier in an amount that does not impair the efficacy of Abies sibirica oil. For example, the food composition may contain at least one of sugar, acid, and sugar alcohol.

The food composition according to one aspect of the present disclosure may be a health food, a functional food, and a food additive composition. The composition can be applied in various formulations, such as tablets, pills, capsules, granules, drinks, caramels, diet bars, tea bags, etc. through a general method including adding various types of excipients or additives. In the composition, in addition to the active ingredients, depending on the formulation or purpose of use, ingredients commonly used in the art may be appropriately selected and mixed by those skilled in the art without difficulty, and a synergistic effect may occur when mixed with other ingredients.

Hereinafter, one aspect of the present disclosure will be described in detail with reference to Examples for specific description. However, Examples according to one aspect of the present disclosure may be modified in various forms, and it is not interpreted that the scope of one aspect of the present disclosure is limited to the following Examples. Examples of one aspect of the present disclosure will be provided for more completely explaining one aspect of the present disclosure to those skilled in the art.

### Example 1. Abies sibirica Oil

Abies sibirica oil, a product from Robertet Co., Ltd. in France, was used.

### Comparative Example 1. Pinus sylvestris Oil

Pinus sylvestris oil was used.

### Experimental Example 1. Observation of Cell Membrane Expression of OR6M1 Gene

In order to overexpress an OR6M1 gene as a fragrance receptor, a plasmid obtained by adding the OR6M1 gene in plasmid cloning DNA (pcDNA) was prepared and used from the Daegu-Gyeongbuk Institute of Science and Technology.

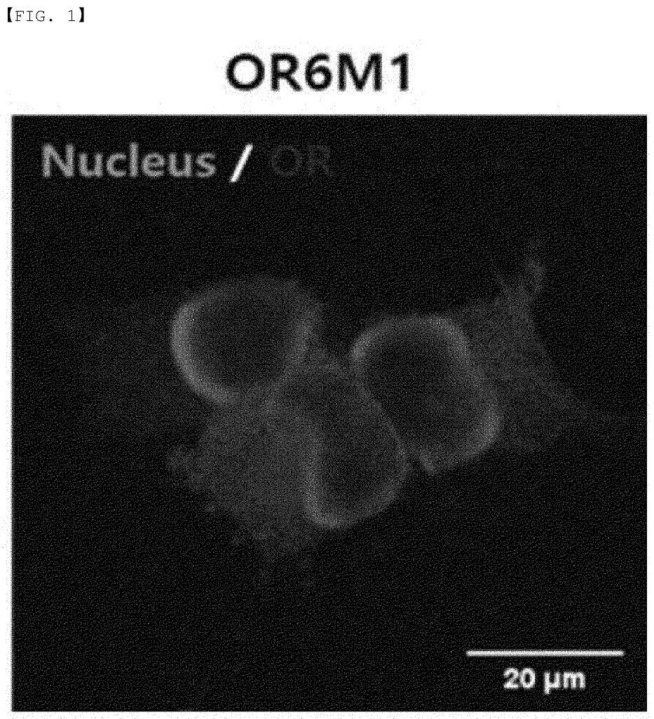

The plasmid containing the OR6M1 gene was injected into HEK293 cells, which were cells without containing the OR6M1 gene, and it was confirmed by immunocytochemistry whether the OR6M1 was normally expressed in the HEK293 cells (FIG. 1).

At this time, anti-FLAG (mouse, Sigma #M1804) was used as a primary antibody, and anti-mouse (Alexa 568, Abcam #ab175472) was used as a secondary antibody, and for imaging, a confocal microscope (LSM700, Zeiss, 400x) was used.

In the results of FIG. 1, red indicated the fragrance receptor OR6M1, and it was confirmed that the OR6M1 was overexpressed in the HEK293 cells.

### Experimental Example 2. Confirmation of Reactivity Between Abies sibirica Oil and OR6M1 Gene

In order to overexpress an OR6M1 gene as a fragrance receptor, a plasmid obtained by adding the OR6M1 gene in plasmid cloning DNA (pcDNA) was prepared and used from the Daegu-Gyeongbuk Institute of Science and Technology.

The plasmid containing the OR6M1 gene was injected into HEK293 cells, which were cells without containing the OR6M1 gene.

Further, in order to overexpress an OR6V1 gene as a fragrance receptor, a plasmid obtained by adding the OR6V1 gene in plasmid cloning DNA (pcDNA) was prepared and used from the Daegu-Gyeongbuk Institute of Science and Technology.

The plasmid containing the OR6V1 gene was injected into HEK293 cells, which were cells without containing the OR6V1 gene.

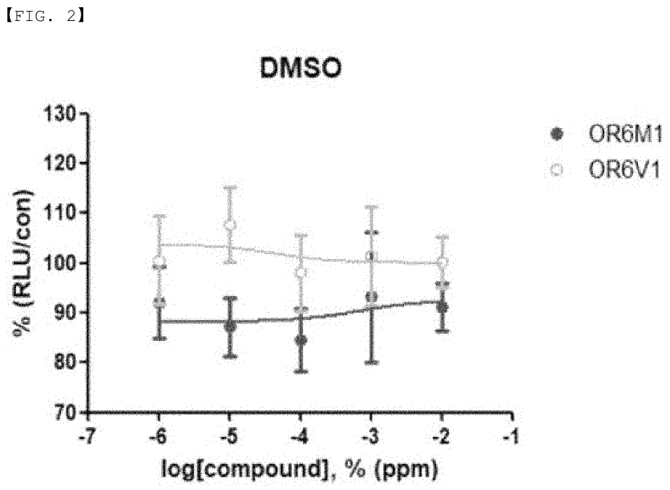

When the HEK293 cells injected with the OR6M1 gene and the HEK293 cells injected with the OR6V1 gene were treated with a DMSO solvent, the concentration of cAMP in each of the HEK293 cells injected with the OR6M1 and OR6V1 genes was not changed (FIG. 2).

By dissolving the Abies sibirica oil prepared in Example 1 in the DMSO solvent, solutions of 0.1 ppm (10⁻³%), 1 ppm (10⁻⁴%), 10 ppm (10⁻³%) and 100 ppm (10⁻²%) were prepared.

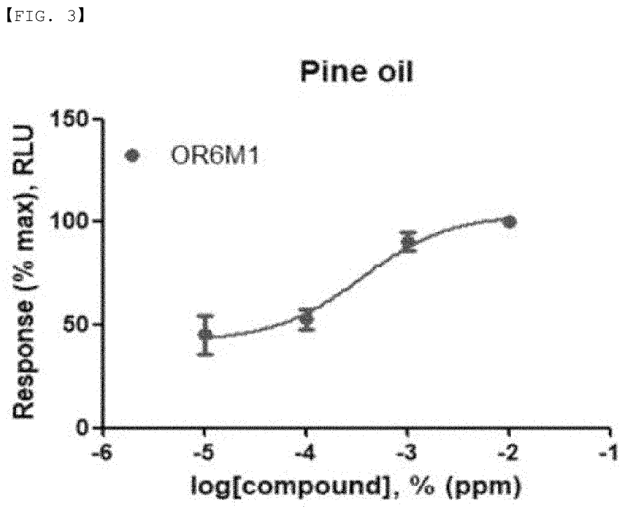

The HEK293 cells injected with the OR6M1 gene and the HEK293 cells injected with the OR6V1 gene were treated with the Abies sibirica oil at the concentrations, respectively, to observe the cAMP concentration of the HEK293 cells (FIG. 3).

As a result, the OR6V1 gene did not react with the Abies sibirica oil, and the OR6M1 gene reacted with the Abies sibirica oil, resulting in increasing the cAMP concentration in the HEK293 cells and depending on the concentration of the Abies sibirica oil.

Thus, it can be seen that the OR6M1 gene as the fragrance receptor reacts selectively with the Abies sibirica oil.

### Experimental Example 3. Measurement of Effect of Restoring Damaged Skin Barrier

An effect of Abies sibirica oil to restore the skin barrier function damaged by the skin damage caused by ultraviolet rays was measured.

Neonatal keratinocytes (normal human epidermal keratinocytes: P988, Ronza) were dispensed into a 60 mm cell culture dish using a keratogenesis medium (KGM) at a density of 1.25×10⁴ cells/dish and then incubated to 80% confluency at 37° C. in a 5% CO₂ incubator.

A 100 ppm (10⁻²%) solution was prepared by dissolving the Abies sibirica oil of Example 1 in a DMSO solvent.

In addition, a 100 ppm (10⁻²%) solution was prepared by dissolving the Pinus sylvestris oil of Comparative Example 1 in a DMSO solvent.

Thereafter, the keratinocytes were treated with ultraviolet rays (UVB 25 mJ/cm²), with both ultraviolet rays (UVB 25 mJ/cm²) and 100 ppm of an Abies sibirica oil solution, and with both ultraviolet rays (UVB 25 mJ/cm²) and 100 ppm of a Pinus sylvestris oil solution, respectively, and incubated for 2 days, and then changes in genes of keratin-1 (KRT1, Hs01549614_g1) and keratin-(KRT10, Hs01043114_g1) as epidermal differentiation markers were confirmed.

The changes in the expression of each epidermal differentiation marker were confirmed by removing a cell growth medium, adding 1 mL of Trizol (Invitrogen), separating RNA according to an RNA separation method of Invitrogen, and then quantifying RNA using an ultraviolet tester (HEWLETT PACKARD) at 260 nm and performing reverse transcription-polymerase chain reaction (RT-PCR). For gene analysis of KRT1 and KRT10 for each sample, taqman probes (Hs01549615_g1, and Hs00166289_m1) were used, and corrected based on a complementary gene RPL13A (Hs01578912_m1).

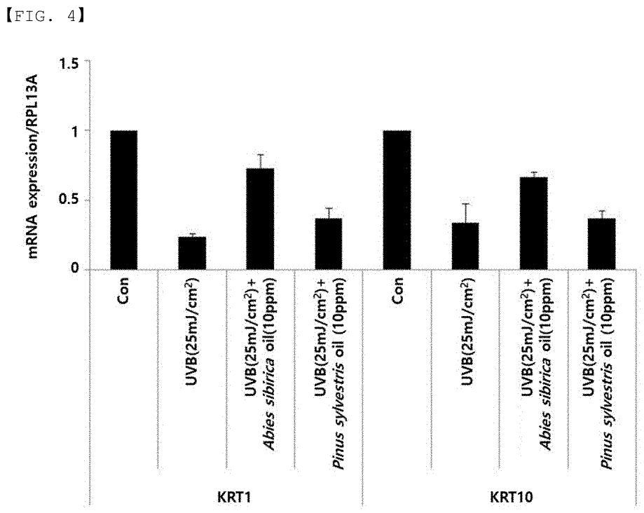

As a result, it was confirmed that the mRNA expression level of KRT1 treated with only ultraviolet rays was significantly reduced based on the mRNA expression level of KRT1 in keratinocytes not treated with ultraviolet rays. However, the mRNA expression level of KRT1 treated with both ultraviolet rays and Abies sibirica oil was higher than that of KRT1 treated with only ultraviolet rays, and from the results, it can be seen that the Abies sibirica oil exhibits an effect of restoring the damaged skin barrier. The mRNA expression level of KRT1 treated with both ultraviolet rays and Pinus sylvestris oil was measured slightly higher than the result treated with only ultraviolet rays, but was measured very lower than the result of treatment with both ultraviolet rays and Abies sibirica oil. In addition, the result of KRT10 was also measured similarly to that of KRT1 (FIG. 4).

From the results of Experimental Examples 1 to 3, it can be seen that since the keratinocytes have the fragrance receptor OR6M1, the Abies sibirica oil reacts with OR6M1 of cells to restore the damaged skin barrier and the Pinus sylvestris oil does not react with the OR6M1 not to restore the damaged skin barrier.

Therefore, it can be seen that the Abies sibirica oil has an effect of enhancing the skin barrier.

### Formulation Example 1. Face Lotion

A face lotion was prepared by a general method according to a composition shown in Table 1 below.

TABLE 1

| Name of raw material | Content (wt %) |
|---|---|
| Glycerin | 3 |
| Butylene glycol | 3 |
| Propylene glycol | 3 |
| Carboxyvinyl polymer | 0.1 |
| Abies sibirica oil of 1 | |
| Example 1 | |
| Beeswax | 4 |
| Polysorbate 60 | 1.5 |
| Caprylic/Capric | 5 |
| Triglyceride | |
| Squalane | 5 |
| Solvitan sesquioleate | 1.5 |
| Cetearyl alcohol | 1 |
| Triethanol amine | 0.2 |
| Preservative, Fragrance | Suitable amount |
| Purified water | Residual |
| Total | 100 |

### Formulation Example 2. Nourishing Lotion

A nourishing lotion was prepared by a general method according to a composition shown in Table 2 below.

TABLE 2

| Name of raw material | Content (wt %) |
|---|---|
| Purified water | Residual |
| Glycerin | 3 |
| Butylene glycol | 3 |
| Liquid paraffin | 5 |
| Beta Glucan | 7 |
| Abies sibirica oil of 1 | |
| Example 1 | |
| Carbomer | 0.1 |
| Caprylic/Capric | 3 |
| Triglyceride | |
| Squalane | 5 |
| Cetearyl glucoside | 1.5 |
| Sorbitan stearate | 0.4 |
| Polysorbate 60 | 1.5 |
| Preservative | Suitable amount |
| Fragrance | Suitable amount |
| Pigment | Suitable amount |
| Triethanol amine | 0.1 |
| Total | 100 |

### Formulation Example 3. Nourishing Cream

A nourishing cream was prepared by a general method according to a composition shown in Table 3 below.

TABLE 3

| Name of raw material | Content (wt %) |
|---|---|
| Glycerin | 3.5 |
| Liquid paraffin | 3 |
| Beta Glucan | 7 |
| Carbomer | 7 |
| Abies sibirica oil of 1 | |
| Example 1 | |
| Caprylic/Capric | 1 |
| Triglyceride | |
| Squalane | 3 |
| Cetearyl glucoside | 5 |
| Sorbitan stearate | 1.5 |
| Polysorbate 60 | 0.4 |
| Triethanol amine | 1.2 |
| Preservative, fragrance Suitable amount | |
| Purified water | Residual |
| Total | 100 |

### Formulation Example 4. Preparation of Drug (Patch) for Topical Administration

A drug (patch) for topical administration was prepared by a general method according to a composition shown in Table 4 below.

TABLE 4

| Name of raw material | Content (wt %) |
|---|---|
| Abies sibirica oil of Example 1 | 1 |
| Beta-1,3-glucan | 3 |
| Diethylamine | 0.7 |
| Sodium sulfite | 0.1 |
| Polyoxyethylene lauryl ether (E.0 = 9) | 1 |
| Polyhydroxyethylene cetyl stearyl 1 | |
| ether (Cetomacrogol 1000) | |
| Viscous paraffin oil | 2.5 |
| Caprylic acid ester/capric acid | 2.5 |
| ester (Cetiol LC) | |
| Polyethylene glycol 400 | 3 |
| Polyacrylic acid (Carbopol 934P) | 1 |
| Purified water | Residual |
| Total | 100 |

### Formulation Example 5. Preparation of Tablets

2 mg of the Abies sibirica oil of Example 1, 100 mg of corn starch, 100 mg of lactose, and 2 mg of magnesium stearate were mixed and then tableted by a general tablet preparation method to prepare tablets.

### Formulation Example 6. Preparation of Pills

0.03 g of the Abies sibirica oil of Example 1, 1.5 g of lactose, 1 g of glycerin and 0.5 g of xylitol were mixed and then prepared to be 4 g per 1 pill according to a general preparation method.

### Formulation Example 7. Preparation of Drinks

360 mg of the Abies sibirica oil of Example 1, 10 g of glucose, 0.6 g of citric acid, and 25 g of liquid oligosaccharide were mixed, and then 300 mL of purified water was added to fill each bottle by 200 mL. The bottle was filled and then sterilized at 130° C. for 4 to 5 seconds to prepare drinks.

### Formulation Example 8. Preparation of Caramel Formulation

58 mg of the Abies sibirica oil of Example 1, 1.8 g of corn syrup, 0.5 g of skim milk, 0.5 g of soybean lecithin, 0.6 g of butter, 0.4 g of hydrogenated vegetable oil, 1.4 g of sugar, 0.58 g of margarine, and 20 mg of salt were mixed to prepare caramel formulations.

In the specification, there have been disclosed typical preferred embodiments of the invention and, although specific terms are employed, they are used in a generic and descriptive sense only and not for purposes of limitation, the scope of the invention being set forth in the claims. Numerous modifications and variations of the invention are possible in light of the above teachings. It is therefore to be understood that within the scope of the appended claims the invention may be practiced otherwise than as specifically described.

1. A skin barrier enhancing composition comprising Abies sibirica oil as an active ingredient.

2. The skin barrier enhancing composition of claim 1, wherein the Abies sibirica oil reacts with an OR6M1 gene as a fragrance receptor present in the skin.

3. The skin barrier enhancing composition of claim 1, wherein the Abies sibirica oil is oil of the leaves, roots, stems, fruits or mixtures thereof of Abies sibirica.

4. The skin barrier enhancing composition of claim 1, wherein the Abies sibirica oil is included in an amount of 0.01 wt % to 10 wt % by weight based on the total weight of the composition.

5. The skin barrier enhancing composition of claim 1, wherein the composition has a skin moisturizing enhancing effect.

6. The skin barrier enhancing composition of claim 1, wherein the composition is a cosmetic composition and comprises a cosmetically acceptable carrier, and wherein the cosmetic composition is in form of a cream, emulsion, or lotion.

7. The skin barrier enhancing composition of claim 1, wherein the composition is a pharmaceutical composition.

8. The skin barrier enhancing composition of claim 1, wherein the composition is a food composition.

9. A method for improving the condition or aesthetic appearance of human skin comprising topically applying to skin in need thereof an effective amount of a composition of claim 6,
wherein said improvement is selected from the group consisting of:
(a) treatment, reduction, and/or prevention of fine lines or wrinkles;
(b) improvement in skin thickness, plumpness, and/or firmness;
(c) improvement in skin suppleness and/or softness;
(d) improvement in skin tone, radiance, and/or clarity;
(e) improvement in skin barrier repair and/or function;
(f) improvement in appearance of skin contours;
(g) improvement in skin moisturization;
or any combination thereof.
